(19) United States
(12) Patent Application Publication
(10) Pub. No.: US 2015/0080637 A1
(43) Pub. Date: Mar. 19, 2015

(54) MICROSCOPIC MAGNETIC COILS FOR NEURAL STIMULATION

(71) Applicant: The General Hospital Corporation, Boston, MA (US)

(72) Inventors: Giorgio Bonmassar, Boston, MA (US); Wim Vanduffel, Charlestown, MA (US)

(21) Appl. No.: 14/486,267

(22) Filed: Sep. 15, 2014

Related U.S. Application Data
(60) Provisional application No. 61/878,241, filed on Sep. 16, 2013.
(60) Provisional application No. 61/889,146, filed on Oct. 10, 2013.

(51) Int. Cl. A61N 2/00; A61N 2/02

(52) U.S. Cl. CPC A61N 2/006; A61N 2/02
USPC 600/14

(57) ABSTRACT

Designs of magnetic microcoil neural stimulator and driving pulse parameters to maximize current in tissue to excite neurons and to harvest energy drained from microcoil. Judiciously designed microcoil stimulator facilitates spatial selectivity and steerability stimulation while lowering consumption of energy and recovering unused energy stored in the coil. Several different coil array layouts for different stimulation strategies are presented.

### CROSS-REFERENCE TO RELATED APPLICATIONS

This patent application claims priority from and benefit of U.S. Provisional Patent Application No. 61/878,241 filed on Sep. 16, 2013 and titled “Microscopic Magnetic Coils For Neural Stimulation”, and U.S. Provisional Patent Application No. 61/889,146 filed on Oct. 10, 2013 and titled “Microscopic Magnetic Coils For Neural Stimulation”. The disclosure of each of the abovementioned provisional patent applications is incorporated herein by reference.

### STATEMENT REGARDING FEDERALLY SPONSORED RESEARCH OR DEVELOPMENT

This invention was made with government support under Grant Number R21EY020961, awarded by the NEI/NIH. The U.S. government has certain rights in the invention.

### TECHNICAL FIELD

The present invention relates to stimulation of neural tissue and, in particular, to a spatially-selectable stimulation of neural tissue with an array of magnetic microcoils.

### BACKGROUND ART

Microscopic magnetic stimulation (μMS) employs microscopic coils to stimulate locally excitable tissue, for example neural tissue ganglion cells in the retina. The neuronal stimulation is achieved by generating a magnetic flux (that has a large peak norm such as 0.1 Tesla, for example, but that is spatially dispersed in a microscopic region of tissue) via discharging an electric input of about several volts through microscopic coil(s) placed on top of the tissue. If the coil(s) are placed within or in close proximity to the excitable tissue, they can induce, in operation, a spatially localized current gradient sufficient in strength to activate the tissue. The generally time-varying electrical signal (such as a current pulse) applied to the coil(s) may be as high as several amperes with durations of up to several hundreds of μs.

One of the biggest challenges to long term operation of implanted μMS microcoil(s) is the reduction of the power consumption of the μMS system, the goal of which is to make the power consumption comparable with that of the conventionally-used Functional Electrical Stimulation (FES) of neural tissue.

### SUMMARY OF THE INVENTION

Embodiments of the present invention provide a method for operating an array of inductive microelements to affect neural activity of biological tissue. The method comprises disposing the array of multiple magnetic coils at a first distance below about 900 microns from the biological tissue (wherein geometrical parameters of a magnetic coil are chosen to define a peak of norm of current density, formed at a point located at a second distance from the magnetic microcoil when the magnetic microcoil is driven with a driving current a level of which is below about 100 amperes) such that said peak is substantially proportional to an outer diameter of said magnetic microcoil. The method further includes magnetically inducing, in the tissue, an induced current having such peak by passing through said magnetic microcoil the driving current including a driving current pulse having an amplitude and a duration that are defined based on empirical data representing a level of neuronal firing threshold in said biological tissue.

### BRIEF DESCRIPTION OF THE DRAWINGS

The invention will be more fully understood by referring to the following Detailed Description in conjunction with the generally not-to-scale Drawings, of which:

FIG. 1A is a schematic diagram illustrating how μMS coil magnetic field may influence cortical neurons;

FIG. 1B is a diagram illustrating a grid of μMS coils disposed on a surface that follows the curvature of the primary motor cortex of a non-human primate;

FIGS. 1C and 1D show, respectively, populated (with 60 surface mounted coils and on a Teflon substrate with 64 traces 12.5 μm wide) and bare μMS arrays;

FIGS. 2A, 2B, 2C, 2D, 2E, and 2F illustrate different spatial configurations of microcoils in a microcoil array according to an embodiment of the invention. FIG. 2A: figure eight, FIG. 2B: concentric dual toroid, FIG. 2C: Helmholtz, FIG. 2C: bifilar, FIG. 2D: radial rotational field, FIG. 2E: axial rotational field, and FIG. 2F: circular loop;

FIG. 3A shows electrically connected N-turn loop microcoil;

FIG. 3B illustrates the equivalent electrical circuit of the embodiment of FIG. 3A;

FIG. 3C is a plot showing an asymmetric triangular pulse of current in the coil of FIG. 3A that can be used to create two rectangular pulses of the electric field in the vicinity of the μMS in the tissue (one above and one below threshold);

FIG. 3D is a plot showing two rectangular pulses of the electric filed formed by an asymmetric triangular pulse current of FIG. 3D in the microcoil. The second sub-threshold rectangular pulse drains the magnetic energy stored in the microcoil, which is then captured by an energy-harvesting capacitor of FIG. 3B;

FIG. 3E illustrates electronic circuitry for a driver of the microcoil of FIG. 3A with a switch capacitor design configured to capture the energy stored in the microcoil during the sub-threshold pulse of the electric field;

FIGS. 4A and 4B illustrate, respectively, 3D current density distribution and linear distribution of current density along x-axis (or y-axis) for a 400 μm diameter microcoil of an embodiment of the invention;

FIGS. 5A and 5B illustrate, respectively, 3D current density distribution and linear distribution of current density along the z-axis for a 400 μm diameter microcoil of an embodiment of the invention;

FIGS. 6A and 6B illustrate, respectively, 3D current density distribution and linear distribution of current density along x-axis (or y-axis) for a 200 μm diameter microcoil of an embodiment of the invention;

FIGS. 7A and 7B illustrate, respectively, 3D current density distribution and linear distribution of current density along the z-axis for a 200 μm diameter microcoil of an embodiment of the invention;

FIGS. 8A and 8B illustrate, respectively, 3D current density distribution and linear distribution of current density along x-axis (or y-axis) for a 200 μm diameter microcoil of an embodiment of the invention;

FIGS. 9A and 9B illustrate, respectively, 3D current density distribution and linear distribution of current density along the z-axis for a 100 μm diameter microcoil of an embodiment of the invention;

FIGS. 10A, 10B, and 10C are plots showing changes in horizontal (across a plane perpendicular to an axis of a coil) distribution of current density induced in tissue as a function of frequency for 0402 microcoil, 0201 microcoil, and 01005 microcoil (surface mounting technology), respectively;

FIGS. 11A, 11B, and 11C are plots showing changes in horizontal (across a plane perpendicular to an axis of a coil) distribution of core relative magnetic permeability of microcoils with diameters of 400 μm, 200 μm, and 100 μm, respectively;

FIGS. 12A, 12B, and 12C are plots showing changes in horizontal (across a plane perpendicular to an axis of a coil) distribution of norm of the spatial gradient of the electric field induced by a embodiment of the coil having 400 μm, 200 μm, and 100 μm outer diameter, respectively;

FIG. 13A illustrates schematically a finite-element model of a seven-turn, spiral coil atop a slab of tissue, analyzed in the present invention;

FIG. 13B is a diagram of the square spiral conductor corresponding to the model of FIG. 13A with lines of magnetic flux and electric potential created with the use of same. The magnetic flux enters the slab of tissue and is confined to a lesser degree to the core (as compared to a solenoid coil).

### DETAILED DESCRIPTION

Embodiments of the invention address the issue of reduction of the power consumption of the μMS system by implementing (a) increase of the gradient of induced current density via scaling down the size of a μMS coil, (b) increase of the slew rate of a coil-driving pulse, (c) the use of coil(s) with high permeability core materials, and (d) the optimization of the shape of the waveform of the coil-driving stimulus.

Although electrical techniques for neuronal stimulation have proven quite useful in some cases, embodiments of this invention are directed to a μMS device and method because, as discussed below, such device and method have several advantages over the electrical stimulation approach. In order for an electrode pair to generate current it needs to be placed in contact with a conductive media (such as the tissue to be excited, or excitable tissue). However, a coil can induce a current at a target location from a distance (for example, through an insulation layer). Embodiments of the microcoil-based stimulator device of the present invention demonstrate that such distance separating the coil(s) and the excitable, target tissue scales approximately with the diameter of the coil.

In electrical stimulation, a bipolar electrode pair (referred to as the ‘cathode’ or source) injects current and depolarizes it, or making the neuronal cell's membrane potential more positive or less negative that may generate an action potential. Whereas in the ‘anode’ or sink the axon membrane potential is hyperpolarized, to the opposite of depolarized, making the membrane potential of the neuronal cell more negative potential that can arrest the neural action potential propagation. The principal issue in the design of electrical stimulation pulses is that charges may accumulate over time and produce electrolysis byproducts capable of damaging or inflaming the tissue. In direct contrast to this, neither sinks nor sources are present when current J is induced by a magnetic field. J is a rotating field that “mirrors” the direction of current direction in the coil. The lack of such “net sources or sinks” derived from the Maxwell Equations ensures that the use of μMS does not ever lead to a net charge buildup. The discussion below is focused on solving Eddy currents under the quasi-static Maxwell equation approximation.

There are several advantages of using the μMS over traditional Functional Electrical Stimulation (FES). For example, MRI examination of Deep Brain Stimulation (DBS) patients can result in neural tissue damage due to excessive heating in the stimulating electrodes used in FES. In this case, the Joule heat is caused by the combination of high RF current density J and high impedance interface that is located between the electrode and the tissue. With the use of μMS, on the other hand, Joule heating effect is greatly attenuated by the use of proper dielectric coating formed around the coil. In addition, direct contact between the metal electrode used in FES and tissue in FES may cause inflammation and glial scarring around the stimulating electrode, which eventually can increase the electrode impedance and stimulation threshold. Although the inflammatory process can be connected with a number of possible causes, one primary mechanism of inflammation involves the water-metal reduction (expressed, for example, as 2Me+3H₂O=Me₂O₃+3H₂) at the conductor/tissue interface, which modifies the local pH value and may cause inflammation in the surrounding tissue. In contrast, such immune reaction can be avoided in μMS procedure by using biocompatible dielectrics. Furthermore, unlike the FES, any realistic pulse in magnetic stimulation process does not lead to charge accumulation in the surrounding tissue. In addition, the penetration of the magnetic field in the surrounding tissue is, in general, different from that of the electric field. Potentially, therefore, the combination of bother the electric and magnetic fields can be used to improve the control of neural stimulation.

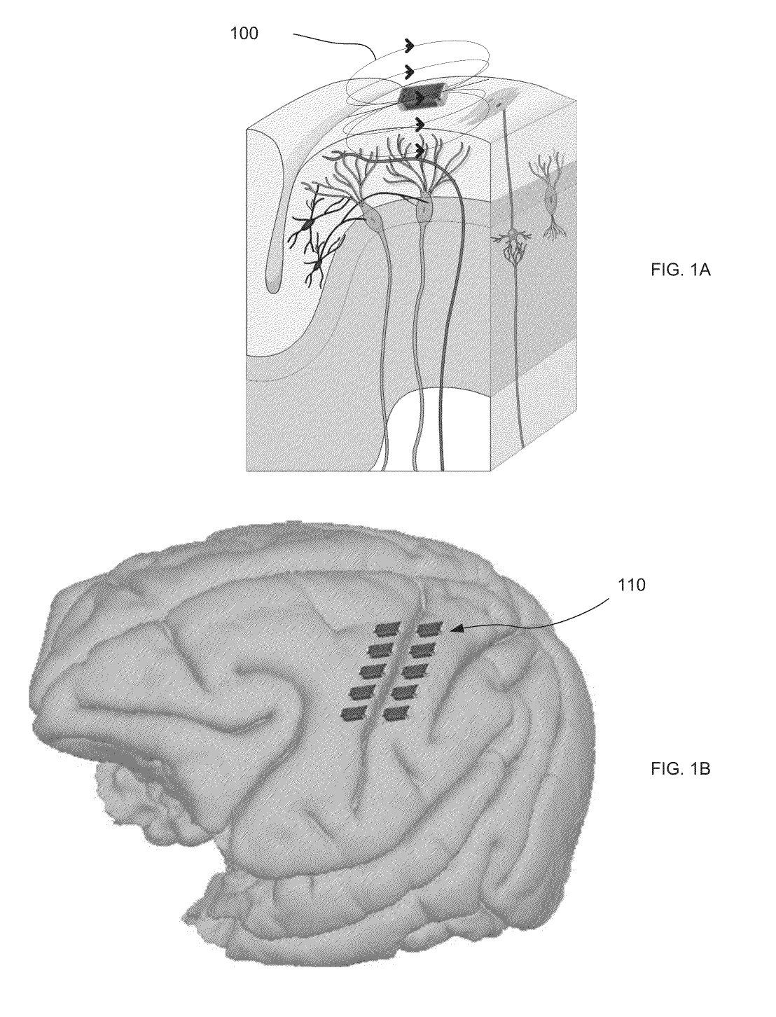

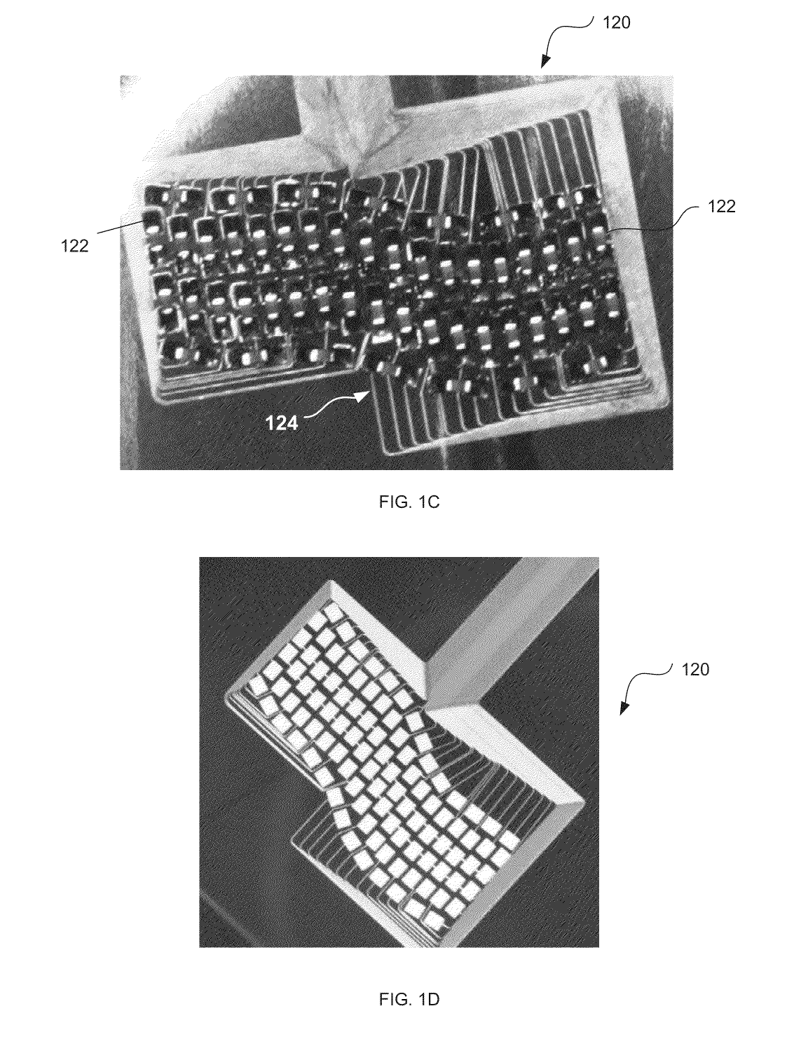

The use of the μMS is also advantageous over the use of transcranial magnetic stimulation (TMS). Unlike the TMS, which requires special bulky custom-made instrumentation for the charge/discharge of large capacitors in order to generate currents on the order of a kA, μMS procedure can be driven by a small class D amplifier. According to the predictions of various activation models (such as that discussed by Warman et al., in “Modeling the effects of electric fields on nerve fibers: determination of excitation thresholds”, in IEEE Transactions on Bio-Medical Engineering, v. 39, no. 12, pp. 1244-54, December 1992), the neural stimulation is primarily cause by the gradient of the electric field (rather than its strength). In reference to FIG. 1A, the μMS procedure can be configured to enable very spatially localized field 100 and, therefore, focused stimulation as the coil(s) can be placed very close to the target tissue (such as cortical tissue or even neuronal cell body or axon) thereby increasing the gradient of induced current density in the tissue and, therefore, the stimulation efficiency. FIG. 1B illustrates, schematically, a layout of an array of magnetic microcoils 110 that follows the curvature of the primary motor cortex. FIG. 1C illustrates a μMS array 120 of microcoils including sixty 0402-SMD coils 122 on a Teflon substrate (with 64 traces 124, each about 12.5 μm wide). FIG. 1D shows the array 120 in a different view.

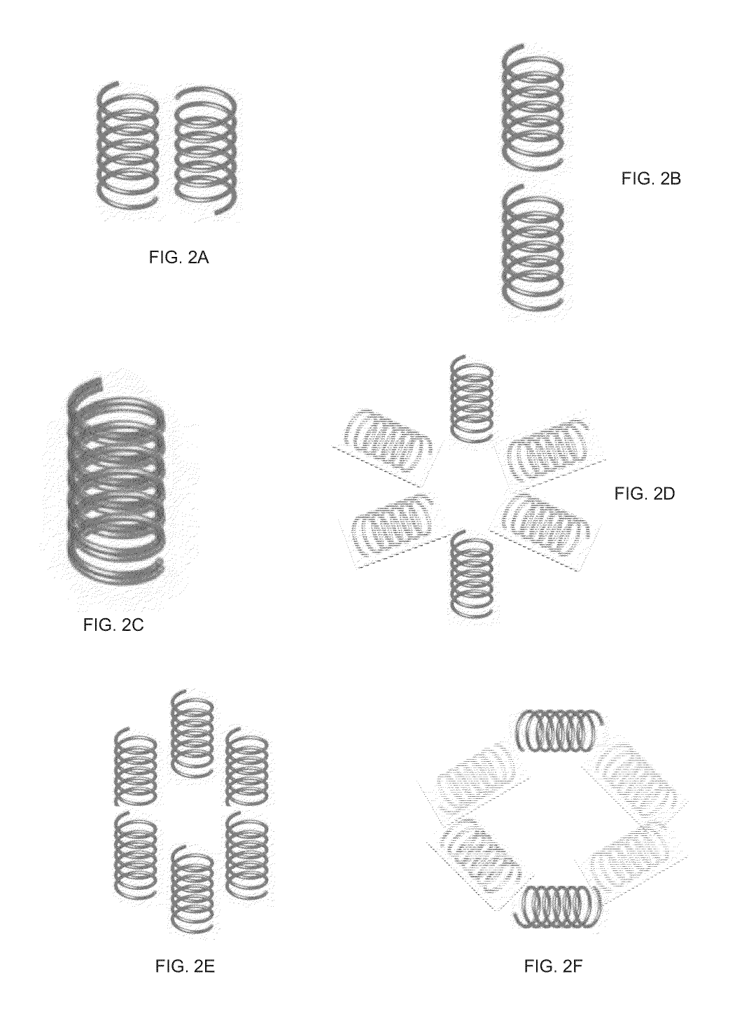

In reference to FIGS. 2A, 2B, 2C, 2D, 2E, and 2F, the magnetic microcoils can be used individually or as a set of multiple microcoil grouped is different spatial configurations to judiciously modify the spatial distribution of the magnetic flux. The “figure eight” spatial configuration formed, as shown in FIGS. 2A and 2B with two or more coils, along with a simple circular element is the most commonly used coil type in TMS to form a more focused and directed stimulus input under the center of the coil. A type of “Helmholtz/Maxwell” coil, shown in FIG. 2C, was used to create uniform magnetic fields for the stimulation of biological tissue and cells in a large coil. Concentric dual toroid spatial coordination of individual coils (shown in FIGS. 2D and 2E) is used in advanced pulsed magnet design and allows to apply different waveforms to each of the coils to optimize magnetic flux, mechanical vibration, and tensile radial stress. The ability to spatially steer the generated magnetic field without the motion of the coil(s) themselves is of paramount importance, since microscopic magnetic stimulation is highly sensitive to directionality of the magnetic field. Therefore, various arrays of coils, see FIG. 2F, may be used for spatial steering of the resulting magnetic field in a required direction by controlling the pulse voltages of each coil.

Neural tissue can be stimulated by passing a short pulse of current through magnetic coil(s). Indeed, such current input generates a time-varying magnetic field B inside and in the space surrounding each coil. In the brain tissue, the time-varying magnetic field B in turn generates an orthogonal electric field E capable of evoking neuronal potentials, according to Faraday's Law as: ∇×E=−∂B/∂t. Neuronal processes (such as axons, for example) that are parallel to the direction of the current density J are depolarized or hyperpolarized depending on the direction and strength of J, but the processes transverse to the direction of J are not affected. Thus, the μMS has a capacity for activating or inhibiting neurons in a spatially-oriented fashion with the use of energy stored in the magnetic field. An inductor (such as a magnetic coil) is an ideal magnetic field generator, and it stores the magnetic field energy W generated by the supplied electric current i. The maximum energy that can be stored in the magnetic field of an ideal inductor is:

$$W = \frac{1}{2} {\int \int \int }_{Q} J ( x , y , z ) \cdot A ( x , y , z ) d x d y d z = \frac{1}{2} {Li}^{2} \qquad ( 1 )$$

where A is the magnetic potential; B=∇×A). In a real inductor, the portion of energy W lost is available to elicit neuronal activity even though the loss reduces the Q-factor or the efficiency and the inductance of the coil.

Power Estimation.

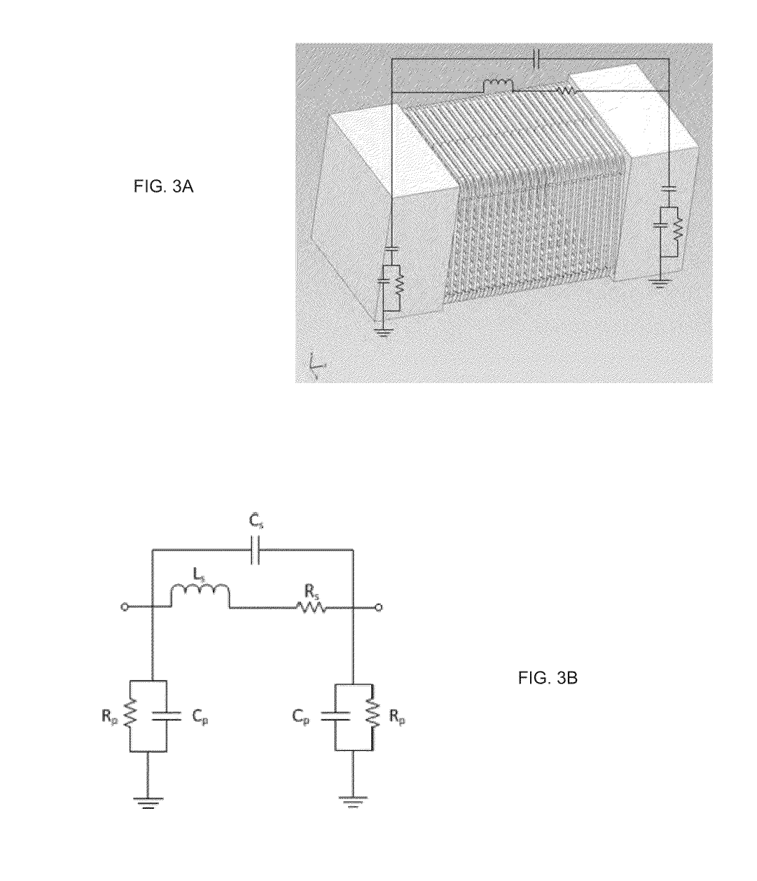

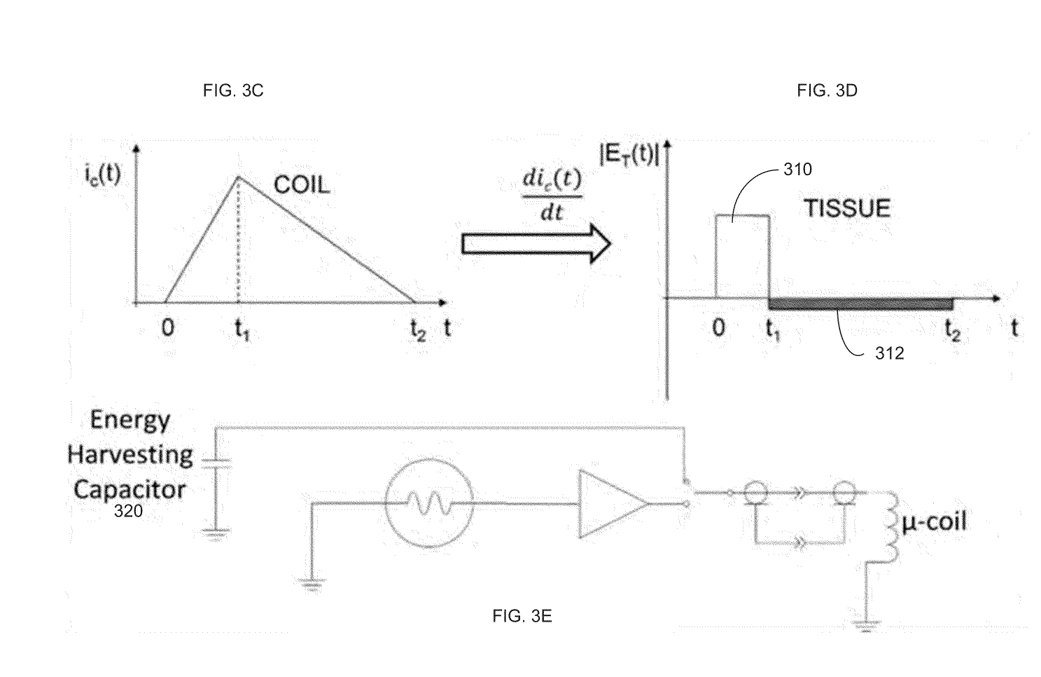

In reference to FIG. 3A, showing schematically electrical connections with N-turn square loop coil and FIG. 3B, showing the equivalent electrical circuit that takes into account different sources of power losses, the power can be estimated by considering the current equivalent-circuit inductor as a lumped circuit. The inductor is modeled as a series resistance Rₛ and inductance Lₛ. Rₛ is generated by the Eddy current effect when the loops of the coil are subjected to time-varying magnetic fields, and is governed by Faraday's law. It is appreciated that, in practice, Eddy currents are relevant only if the thickness of the conducting element of the coil is substantially greater than the skin depth:

$$δ = \frac{1}{πσμ f} \qquad ( 2 )$$

where ρ is the resistivity of conducting element of the coil [Ω-m], μ the permeability [H/m] and f the frequency [Hz]. The bridge between the center of the spiral and one of the terminals generates direct capacitive coupling between the two terminals of the inductor, modeled by a feedthrough capacitance Cₛ.

The capacitance (usually an oxide) between the spiral and the substrate is modeled by a capacitance Cₒₓ. The capacitance and resistance of the silicon substrate are modeled by a parallel of a resistance R_{sub} and a capacitance C_{sub}.

This equivalent circuit can be used to evaluate the complex power [VA] in the spiral magnetic coil, expressed in the frequency domain as a function of impedance Z_{c}(ω) of the N-turn square-loop coil as follows:

$${P}_{C} = \frac{1}{2} {\int }_{- ∞}^{+ ∞} {\vert I ( ω ) \vert}^{2} \underbrace{[ ( \frac{1}{jω {C}_{p}} // {R}_{p} ) // ( ( \frac{1}{jω {C}_{s}} // ( jω {L}_{s} + {R}_{s} ) ) + ( \frac{1}{jω {C}_{p}} // {R}_{p} ) ) ]}_{{Z}_{c} ( ω )} d ω \qquad ( 3 )$$

In the above, the notation Rᵢ∥Rⱼ represents that Rᵢ is parallel to Rⱼ, the result of which is calculated as known in the art.

Coil Heating.

The power dissipated by the coil when driven by a train of pulses is:

P_{c}=Nₛ∫_{B}|I(ω)|²Re(Z_{c}(ω))dω (4)

where Nₛ is the number of pulses per second, Z_{c} (ω) is the complex impedance of the coil and B is the frequency band of the pulse response. The quantity P_{d} represents the amount of power that is converted in heat through the process of Joule heating, by which the passage of an electric current through a conductor releases heat. The heating can be computed by solving the partial differential heat equation.

Induced Currents.

The general form of the Ampere's equation (that includes the displacement currents and is based on the assumption of time-harmonic fields) is:

∇×H=(jω∈₀∈ᵣ+σ)E+J^{e} (5)

where J^{e} is the current that flows in the μMS coil(s), and a is the conductivity of any conductive material. The constitutive relations of the material and the definition of the magnetic potential A are:

$$\begin{matrix}H = \frac{B}{{μ}_{0} {μ}_{r}} & ( 6 ) \\ B = ∇ \times A & ( 7 )\end{matrix}$$

The electric field formed in the tissue in response to B is expressed by Faraday's law:

Eₜ=−jωA−∇ø (8)

where ø is the scalar potential. Here, it is assumed that ∇ø=0 because in an unbounded medium, ø is only due to free charges and no such sources are present. From Eqs. (5), (6), (7), (8) the time-harmonic equation of the Maxwell-Ampere's law can be derived as:

$$( jωσ - {ω}^{2} {ɛ}_{0} {ɛ}_{r} ) A + ∇ \times ( \frac{1}{{μ}_{0} {μ}_{r}} ∇ \times A ) = {J}^{e} \qquad ( 10 )$$

In the cylindrical coordinates (r, z, φ), where the axis of a microcoil coil is perpendicular to the rz plane, and when each turn or loop of the coil can be approximated by a circle with radius r and potential Vᵣ, the external current density has norm:

$${J}^{e} = \frac{σ {V}_{r}}{2 π r} {u}_{ϕ} \qquad ( 11 )$$

where φ∈[0; 2π] and y_{φ} is the unit vector in the φ-direction inside the coil and zero everywhere else.

The current induced in the tissue, then, can be found by solving numerically the following quasistatic equation:

$$( jωσ - {ω}^{2} {ɛ}_{0} {ɛ}_{r} ) A + ∇ \times ( \frac{1}{{μ}_{0} {μ}_{r}} ∇ \times A ) = ( \frac{σ {V}_{r}}{2 π r} ) {u}_{ϕ} \qquad ( 12 )$$

Pulse Design.

The frequency response of the coil equivalent circuit of FIG. 3B can be expressed, in the following Eq. (13), as:

$$\begin{matrix}I ( ω ) = \frac{V ( ω )}{{Z}_{C} ( ω )} \\ = \frac{V ( ω ) ( \begin{matrix}{R}_{p} + {R}_{s} + {R}_{p}^{2} {R}_{s} + \\ jω ( {L}_{s} + {R}_{p}^{2} {C}_{p} + 2 {R}_{p} {R}_{s} {C}_{p} + {R}_{p} {R}_{s} {C}_{s} ) - \\ {ω}^{2} ( 2 {R}_{p} {C}_{p} {L}_{s} + {R}_{p} {C}_{s} {L}_{s} + {R}_{p}^{2} {R}_{s} {C}_{p}^{2} + {R}_{p}^{2} {R}_{s} {C}_{p} {C}_{s} ) - \\ {jω}^{3} ( {R}_{p}^{2} {C}_{p}^{2} {L}_{s} + {R}_{p}^{2} {C}_{p} {C}_{s} {L}_{s} )\end{matrix} )}{( \begin{matrix}1 + {R}_{p} {R}_{s} + ωj ( {R}_{p} {C}_{p} + {R}_{p} {L}_{s} + {R}_{s} {C}_{s} ) - \\ {ω}^{2} {C}_{s} {L}_{s} - {ω}^{2} {R}_{p} {R}_{s} {C}_{p} {C}_{s} - {jω}^{3} {R}_{p} {C}_{p} {C}_{s} {L}_{s}\end{matrix} ) ( 1 - jω {R}_{p} {C}_{p} )} \\ \approx \frac{jω Cs ( Rs + jω Ls )}{Rs + jω ( Cs + Ls )} V ( ω )\end{matrix} \qquad ( 13 )$$

The optimal coil-driving pulse of current is the one that allows for creation of maximum electric field in the coil-surrounding tissue to excite neurons and, at the same time, minimizes the energy and/or power consumption to both reduce the microcoil heating and increase the battery life, thereby creating conditions for optimization of long term implantation of the coil. The energy is dissipated into heat mostly through the resistance Rₛ and therefore is wasted and it may be absorbed by the tissue and dispersed through perfusion. With the approximation assumption is that parallel stray impedances due to packaging are negligible or that Rₚ<<Rₛ and C_{c}<<Cₛ. The ideal pulse is one that provides a maximal electric field in the tissue with which to excite neurons while minimizing the energy, thus improving microcoil battery life in the context of long-term implantation. The electromotive force in the tissue is proportional to the first time-derivative of the current flowing in the coil, and in frequency space it can be expressed as

Eₜ=−jωA∝−jωI(ω) (14)

The optimal current stimulation is the pulse i (t) which minimizes the total complex power and maximizes di(t)/dt, and which in frequency domain is expressed in the following Eq. (15) as:

$${\int }_{- {ω}_{0}}^{+ {ω}_{0}} {I ( ω )}^{2} {Z}_{c} ( ω ) d ω - jω I ( ω ) {\vert}_{- {ω}_{0}}^{+ {ω}_{0}} = {\int }_{- {ω}_{0}}^{+ {ω}_{0}} [ {I ( ω )}^{2} {Z}_{c} ( ω ) - j \frac{d [ ω I ( ω ) ]}{d ω} ] d ω = {\int }_{- {ω}_{0}}^{+ {ω}_{0}} [ {I ( ω )}^{2} {Z}_{c} ( ω ) - jω {I}^{′} ( ω ) - j I ( ω ) ] d ω = {\int }_{- {ω}_{0}}^{+ {ω}_{0}} [ {I ( ω )}^{2} {Z}_{c} ( ω ) - jω {I}^{′} ( ω ) ] d ω - j {I}_{0} \qquad ( 15 )$$

where I₀∈ [symbol] is the mean of the Hermitian solution I(ω), and the integral functional is minimized by means of calculus of variations [18]:

\tilde{H}(I(ω))=∫_{-ω}_{₀}^{+ω}^{₀}[I(ω)²Z_{c}(ω)−λjωI′(ω)]dω (16)

where ω₀ is the angular frequency of the limits of the symmetrical frequency band and λ is the Lagrangian multiplier.

Pulse Design: Case 1: Case: Z_{c}(ω)=jωLₛ:

In the simplified case of an ideal inductor, the functional dependence for current of Eq. (15) has an extremum only if the following Euler-Lagrange differential equation is satisfied:

1+2ωLₛI(ω)=0 (17)

In this case,

$$I ( ω ) = - \frac{1}{2 ω {L}_{s}} \qquad ( 18 )$$

The time-dependence of such current is represented by a rectangular pulse:

i(t)=C u(αt) (19)

where u(t) is the rectangular (or pulse) function. In practice, both α and C are set experimentally with the use of the neuronal firing threshold level and by the slew rate of the amplifier. Since the inductors are of RF type, α≈1 GHz range, such a short pulse can be implemented in a real world stimulator as a very steep triangle function, which in turns produces (i.e., the time derivative) a rectangular pulse in the tissue.

In practice, a net neuronal activation or inhibition can often be achieved with μMS by driving the coil with a sharp rising edge followed by a slowly falling dip (or vice versa), so that the resulting pulse is asymmetric, thereby producing an induced current pulse in the tissue above threshold in the raising edge followed by a sub-threshold current in the falling edge (or vice versa) (as schematically shown in FIG. 3C). The sub-threshold rectangular pulse 310 is used to drive the coil back to resting state (i.e., zero current in the coil) to minimize power consumption and coil heating, FIG. 3D. Furthermore, this sub-threshold pulse may be used for energy harvesting, since the second sub-threshold pulse 312 drains the coil from the magnetic energy stored, which can be captured by an energy-harvesting capacitor 320, FIG. 3E.

Pulse Design: Case 2: Case: Z_{c}(ω)=jωLₛ+Rₛ

In this case, the Euler-Lagrange differential equation:

2(μs+jωLs)I(ω)+j=0 (20)

results in a frequency-domain solution

$$I ( ω ) = - \frac{1}{2} \frac{j Rs + Ls ω}{{Rs}^{2} + {Ls}^{2} {ω}^{2}} \qquad ( 21 )$$

or a time-domain solution

i(t)=C u(αt)e^{-βt} (22)

where=Rₛ/Lₛ.

Pulse Design: Case 3: Z_{c}(ω)=(jωLₛ+Rₛ)/Cₛ:

In the case when all of the R, L, and C elements are included into consideration, the Euler-Lagrange differential equation related to Eq. (15) is:

$$1 - \frac{jω Cs ( Rs + jω Ls )}{Rs + jω ( Cs + Ls )} I ( ω ) = 0 \qquad ( 23 )$$

with a frequency-domain solution of

$$I ( ω ) = - \frac{1}{2 ω Cs} \frac{Rs - jω ( Cs + Ls )}{ω Ls - j Rs} , \qquad ( 24 )$$

the inverse Fourier Transform of which is:

$$( t ) = \{ \begin{matrix}Cu ( α t ) {e}^{- γ t} ( cos ( {ω}_{d} t ) - \frac{γ}{{ω}_{d}} sin ( {ω}_{d} t ) ) & {ω}_{0} > γ \\ Cu ( α t ) {e}^{- γ t} ( 1 - γ t ) & {ω}_{0} = α \\ Cu ( α t ) {e}^{- γ t} ( cos h ( {ω}_{d} t ) - \frac{γ}{{ω}_{d}} sin h ( {ω}_{d} t ) ) & {ω}_{0} < α\end{matrix} \qquad ( 25 )$$

In practically realistic pulses of current, a net neuronal activation or inhibition can be achieved with μMS by driving the coil with a sharp rising edge followed by a slowly falling dip (or vice versa), so that the resulting pulse is asymmetric producing an induced current pulse in the tissue above threshold in the raising edge followed by a sub-threshold current in the falling edge (or vice versa). The second edge is needed to drive back the coil to resting state which is null to minimize power consumption and coil heating.

Simulations

Methods.

The Finite Element Method (FEM) was used to study power dissipation, heat, induced currents and electric in the μMS coils. The simulations were performed in Multiphysics 4.3a (COMSOL, Burlington Mass.) using a 10 μm accurate modeling of the coil and tissue geometry electromagnetic field and induction heating estimation. The FEM geometry included a cylindrical container of about 3 mm radius and 3 mm in height, which enclosed different objects: a physiological solution, a quartz core surrounded by the copper solenoid, and top bottom copper cylindrical terminals. The FEM calculations were performed for coils with different numbers of coil turns (for example, 10, 15 and 21), stimulation frequencies and core materials inside a uniform volume conducting material representing the physiological solution or neuronal tissue with similar electrical characteristics.

The FEM method was used to numerically solve Eq. (12) with respect to the magnetic potential A in frequency domain at different frequencies of interest (for example, in the range from 100 kHz to 1 MHz) and with different magnetic permeability values (for example, from 1 to 10⁶). The setting of a subdomain or component of the geometry specifies the properties of the materials and the initial conditions for each model. The material considered had the following properties: (copper) σ=5.998·10⁷S/m, ∈ᵣ=1, μᵣ=1,

$$( air ) σ = 0 \frac{S}{m} ,$$

∈ᵣ=1, μᵣ=1, and (physiological solution) σ=3 S/m, ∈ᵣ=30, μᵣ=1.

All the surface boundaries around the outer shell of the cylindrical container were set to n×A=0 (sets the tangential components of the magnetic potential to zero at the outer surface). The initial conditions were all A=0 (i.e., null magnetic potential) in the entire FEM geometry. The mesh used for calculations was a Delaunay set with adaptive refine meshing option and a maximum element size of 220 μm on all domains. The voltage in each turn was the same for all the simulations and was found experimentally to be approximately a value of Vᵣ=4.19 V.

Results.

Below, data associated with the current density distribution inside and around the coils of different sizes (which were designed according to an embodiment of the invention) are discussed in order to characterize how small a coil can be constructed that is still capable of practically stimulating neuronal cells.

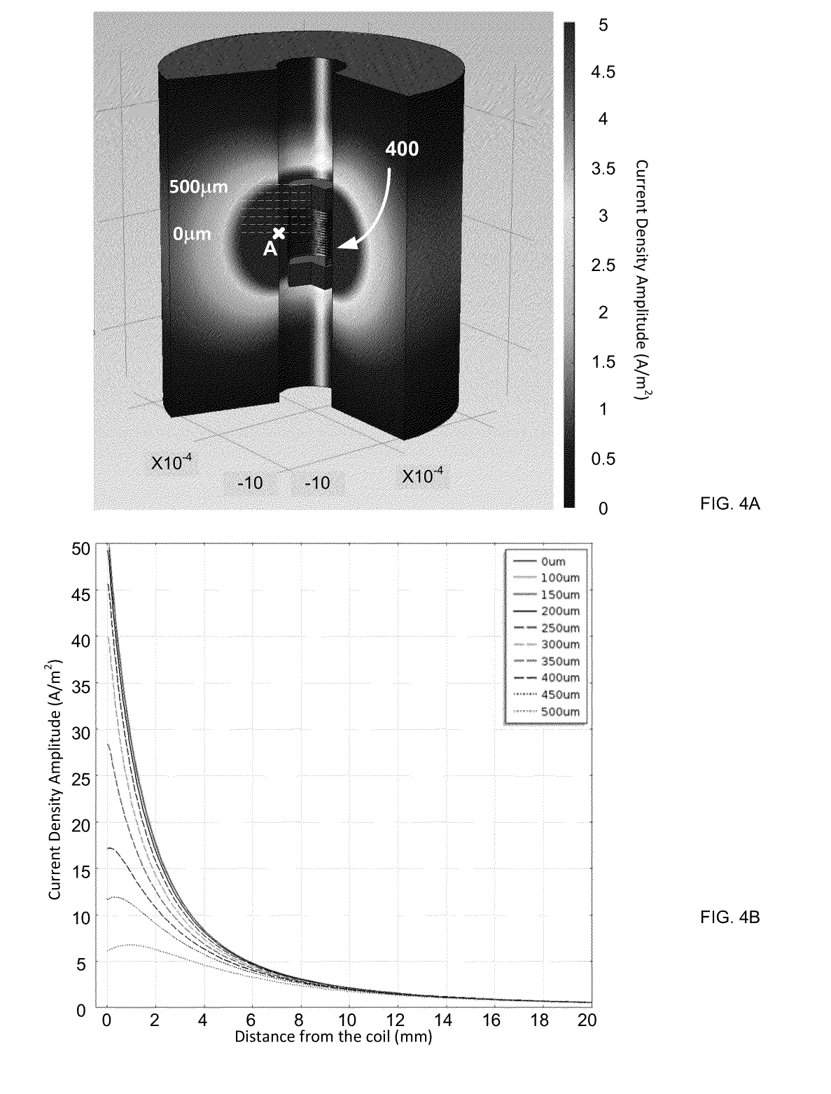

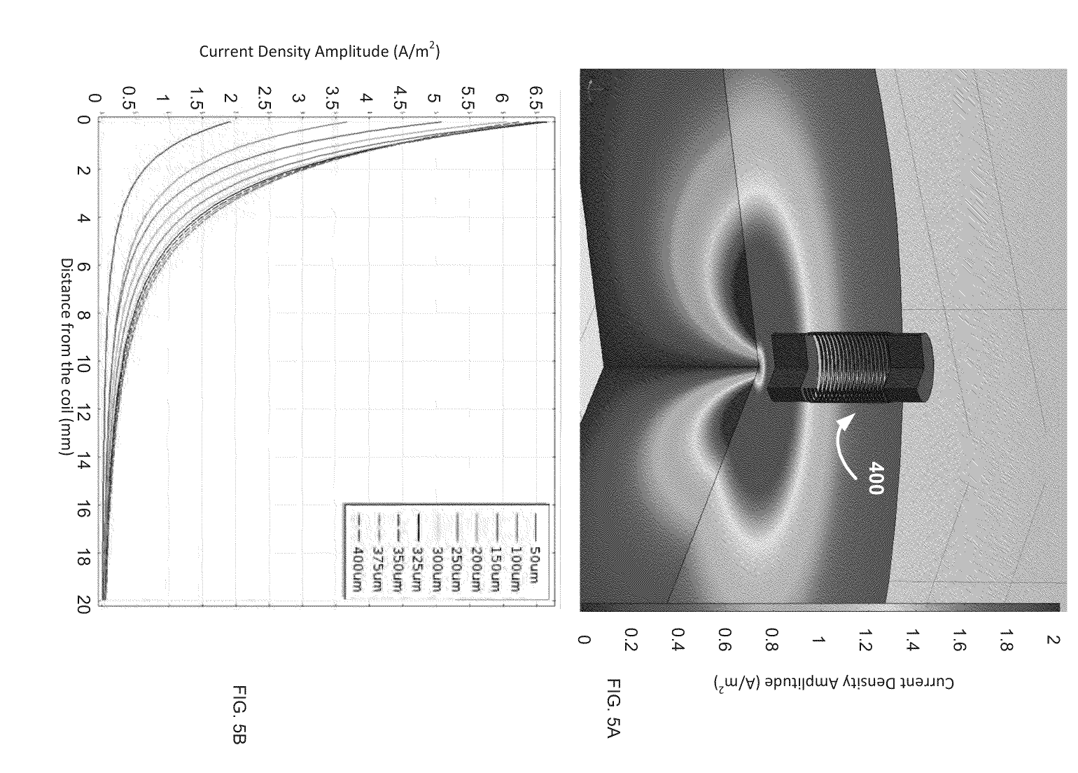

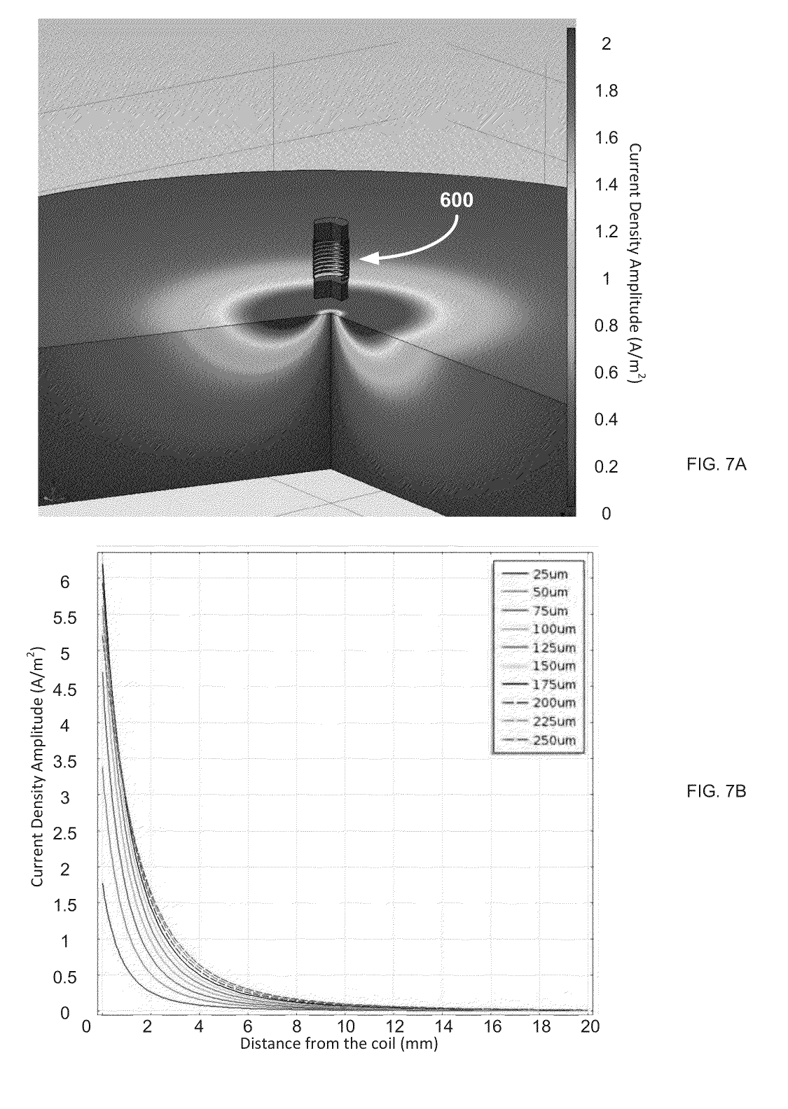

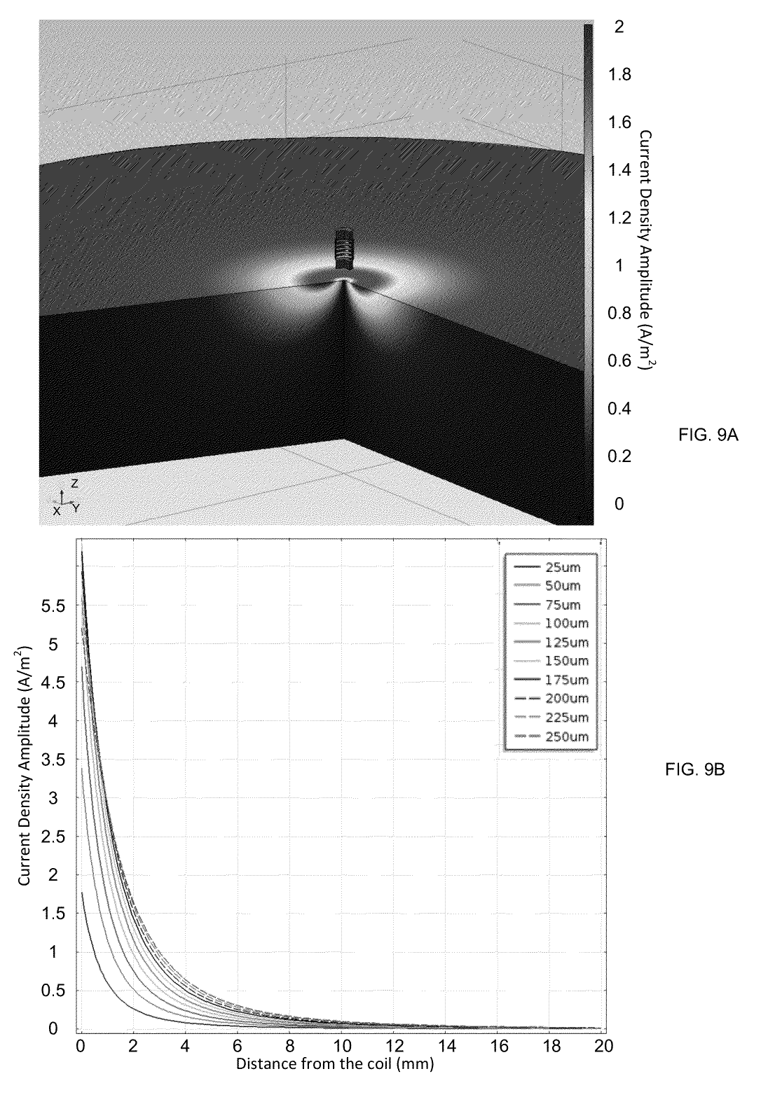

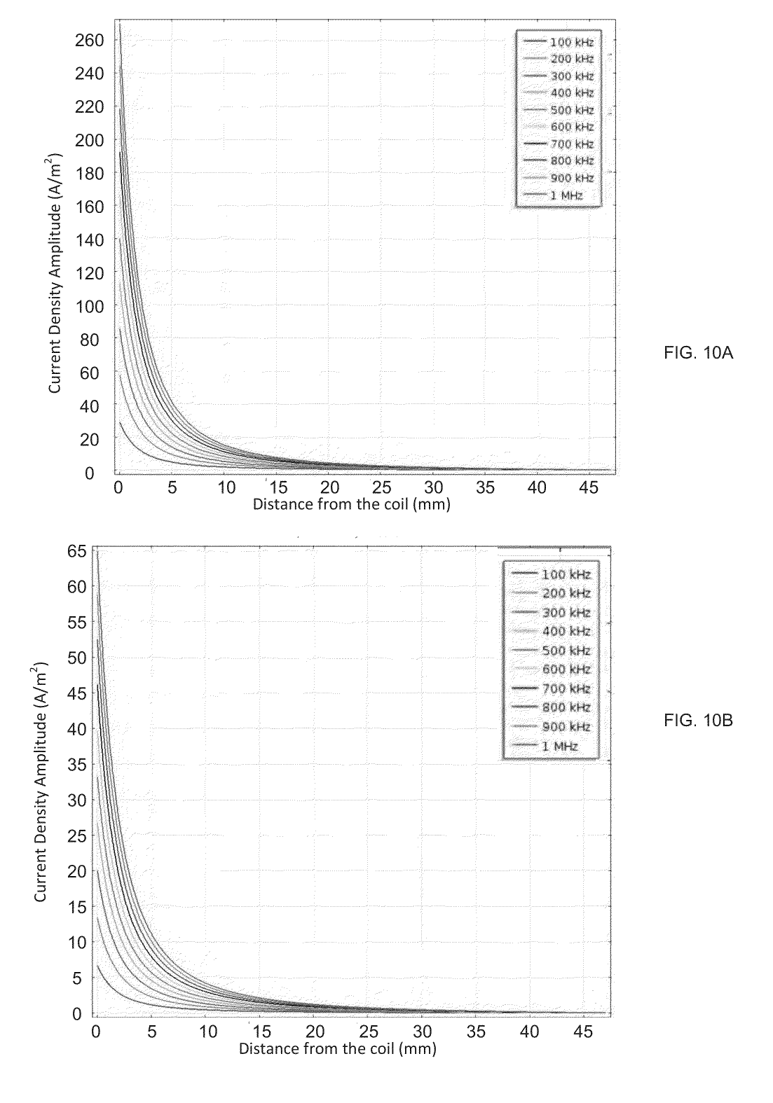

The current density induced by the coil due to Faraday's law varies in the tissue medium space. Current density distributions are presented for the space around the coil (FIGS. 4A, 6A, 8A) and below the coil (FIGS. 5A, 7A, 9A). The current density distribution within the tissue medium changes with the diameter of the different coils studied coils (as follows from the results shown in FIGS. 4A, 5A, 6A, 7A, 8A, and 9A), with higher induced fields adjacent to the coils and lower local maxima fields in proximity of the coil terminals. The distributions of the current densities along the radial lines at different heights (or, put differently, along a radius in different cross-sections of the coil, and shown schematically in FIG. 4A) are nonlinear regardless of the coil size. This is demonstrated in FIGS. 4B, 5B, 6B, 7B, 8B, and 9B. When considering the current density distribution around the coil (FIGS. 4A, 6A, 8A) the current densities have a local maximum in proximity of the minimum distance from the coil. When considering the current density distributions below the terminal (FIGS. 5A, 7A, 9A), all current densities have a local maximum located inside each terminal.

In reference to FIGS. 4A and 4B, 6A and 6B, 8A and 8B, the current density norm distribution increases with the diameter of the different studied coils 400, 600, 800. The current density norm peaks at 50 A/m² is the highest in the case of the coil 400 of FIG. 4A with a 400 μm outer diameter (OD) for currents assessed at a distance of about 100 μm from the windings of the coil 400 at half height of the coil 400. The currents drop to 4.5 A/m² at 100 μm from the outer edge of the coil terminals. At the midpoint, the current density curve was fit to an exponential equation and the coefficients are presented at the top portion of Table 1, with a good quality of fit expressed by R-square/adjusted R-square of 0.9999 and a root-mean-square error (RMSE) of 0.172.

TABLE 1
Coefficients of the exponential function: J (r) = p₁ · exp (r · q₁) +
p₂ · exp (r · q₂) or the distribution current densities perpendicular and in the middle of the coil axis.

| | p₁ | p₂ | q₁ | q₂ |
|---|---|---|---|---|
| | (95% conf bounds) | (95% conf bounds) | (95% conf bounds) | (95% conf bounds) |
| Diameter | A | A/m | m⁻¹ | m⁻² |
| 400 μm | 14.47 (13.8, 15.14) | 36.88 (36.26, 37.5) | −1888 (−1948, −1828) | −8045 (−8203, −7886) |
| 200 μm | 40.44 (39.49, 41.39) | 9.256 (8.247, 10.27) | −1.353 (−1.392, −1.313) 10⁴ | −2824 (−3063, −2584) |
| 100 μm | 35.65 (35.03, 36.28) | 8.953 (8.291, 9.614) | −2.62 (−2.674, −2.565) 10⁴ | −5577 (−5903, −5252) |

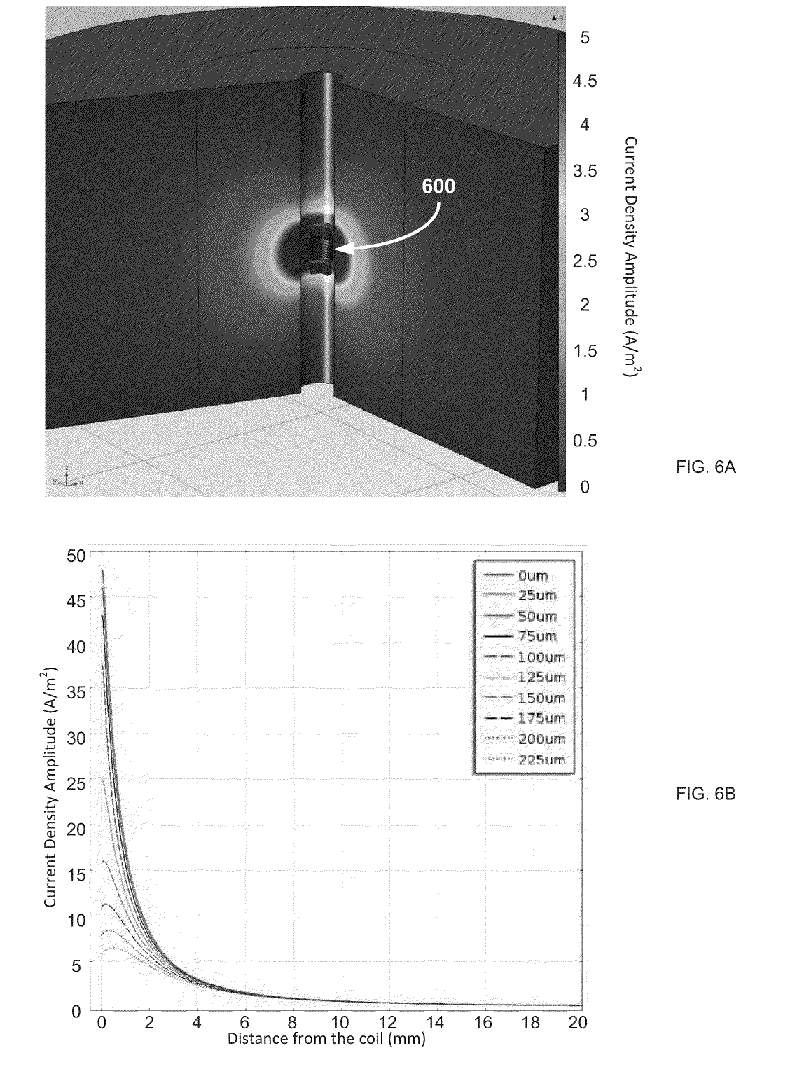

In the case of a 200 μm OD coil 600 of FIG. 6A, as the coil geometry scales down by half in comparison with FIG. 4A, similarly does the peak current density shown in FIG. 6B. The peak current density value assessed at about 100 μm distance from the windings is approximately 25 A/m² at half height of the coil, and drops to about 3 A/m² (which is only about 33% of that corresponding to the coil 400) at 100 μm from the outer edge of the coil terminals. The middle portion of Table 1 presents the current density curve coefficients fit to an exponential function, with a goodness of fit expressed by an R-square/adjusted R-square of 0.9996 and RMSE of 0.2959.

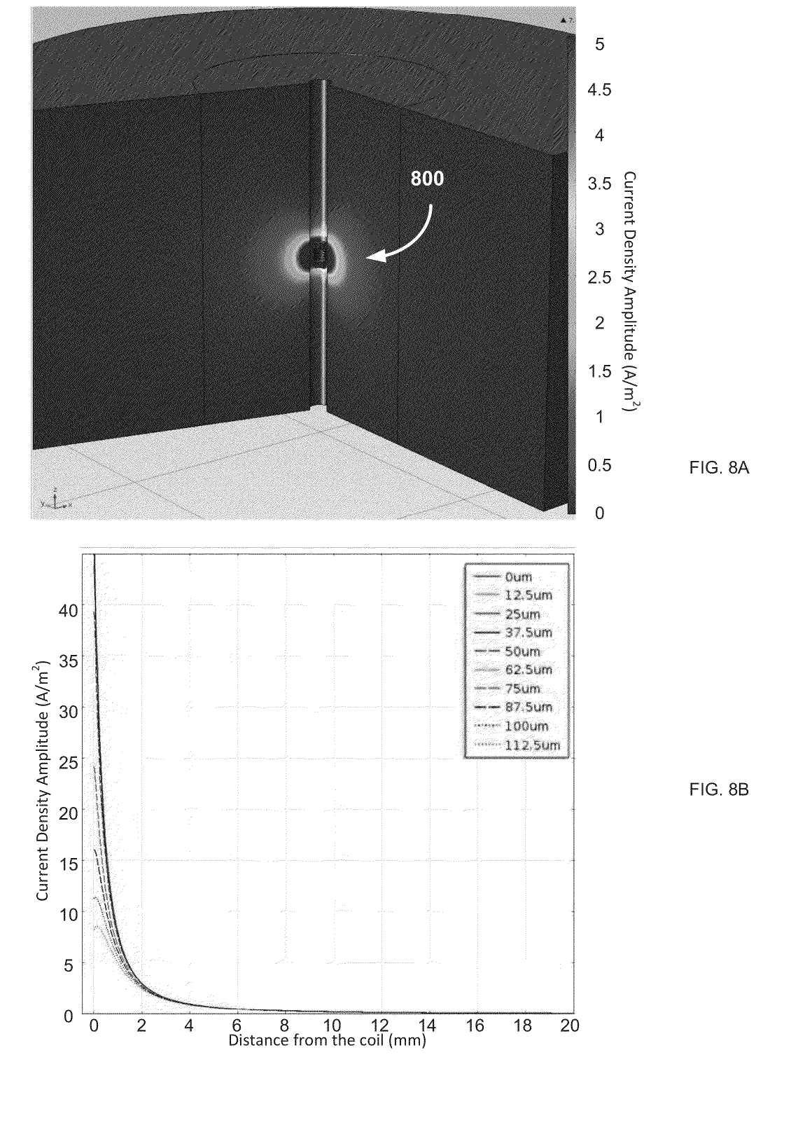

Similarly, in the case of a 100 μm OD coil 800 of FIGS. 8A, 8B, and as the geometry of the coil scales down by half in comparison with the coil 600 of FIGS. 6A, 6B so does the peak current density, shown in FIG. 6B. At 100 μm distance from the winding of the coil 800 the current density is approximately 12.5 A/m² at half height of the coil and drops to about 3 A/m² at 100 μm distance from the outer edge of the coil terminals. Therefore, there is a direct proportionality of change in peak densities with changes in coil diameter at a distance of 100 μm the midway point between the coil terminals (as shown by point A in FIG. 4A). This direct proportionality of change, however, is not maintained at 100 μm from the coil terminals, where between the 400 μm and 200 μm diameter coils 400, 600 there is about a 33% change and a negligible change between the 200 μm and 100 μm diameter coils 600, 800. The current densities norm below each terminal exhibits a similar trend with the size of the coil (as can be seen from FIGS. 5B, 7B, 9B). The coefficients of the current density curve fit corresponding to the midpoint between the coil terminals, are presented in the bottom portion of Table 1, and are characterized by a good fit with R-square/adjusted R-square of about 0.9998 and the RMSE of 0.161.

The distance of neural tissue activation (i.e., the distance this coil μMS activates ganglion cells in the retina at a slightly lower 70 kHz frequency of operation, as defined by Bonmassar et al. in “Microscopic magnetic stimulation of neural tissue,” Nature communications, vol. 3, pp. 921, 2012) for certain peak current density norm scales approximately with the diameter of the coil. For example, the peak for the 400 μm OD coil is about 18 A/m² and occurs for a 300 μm distance of activation, for a 200 μm OD coil the same peak current density norm occurs approximately at 150 μm distance from the coil, and for the 100 μm OD coil—at about 75 μm from the coil.

Effects of Frequency on Current Density Distribution.

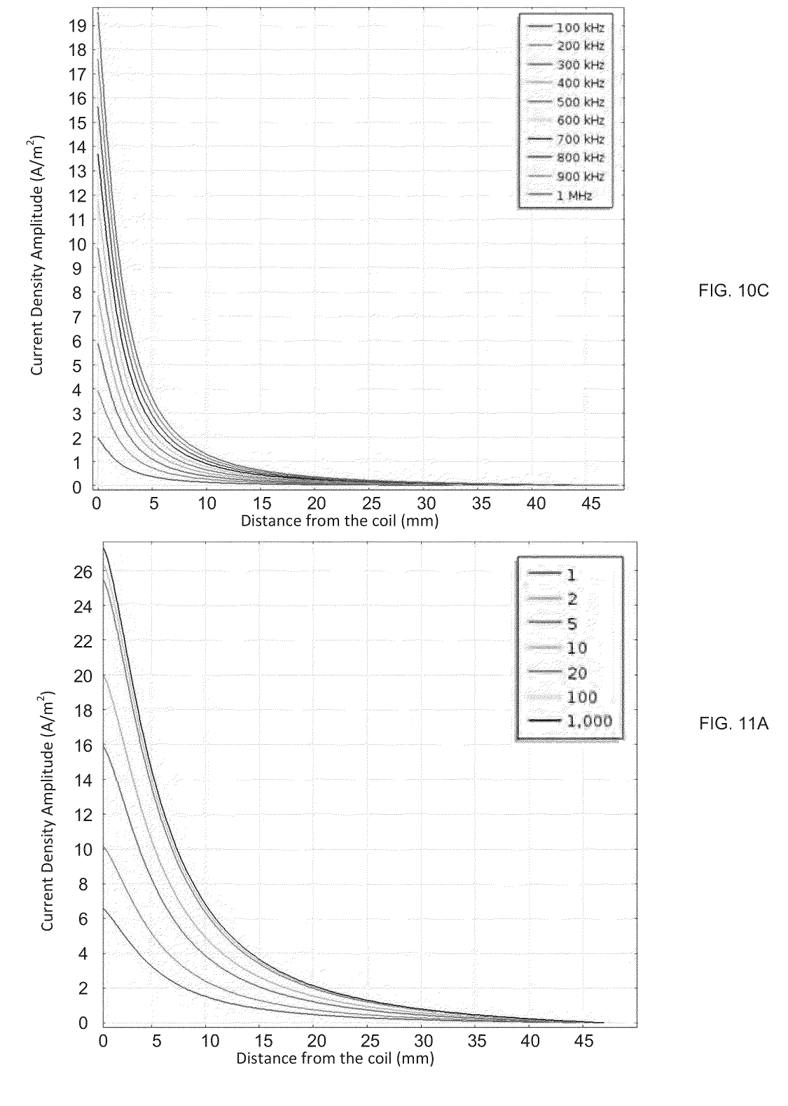

In reference to FIG. 10A, the current densities norm increase linearly with the stimulation frequency, as predicted by Faraday's law of Eq. (8). In particular, the peak of current density at 300 μm distance from the coil axis for the 400 μm OD coil 400 of FIG. 4A changes from about 18 A/m² at 100 kHz of operation to about 36 A/m² at 200 kHz and so on. A similar behavior is demonstrated by the 200 μm OD coil (FIG. 10B) and the 100 μm OD coil (FIG. 10C).

Effects of Permeability on Current Density Distribution.

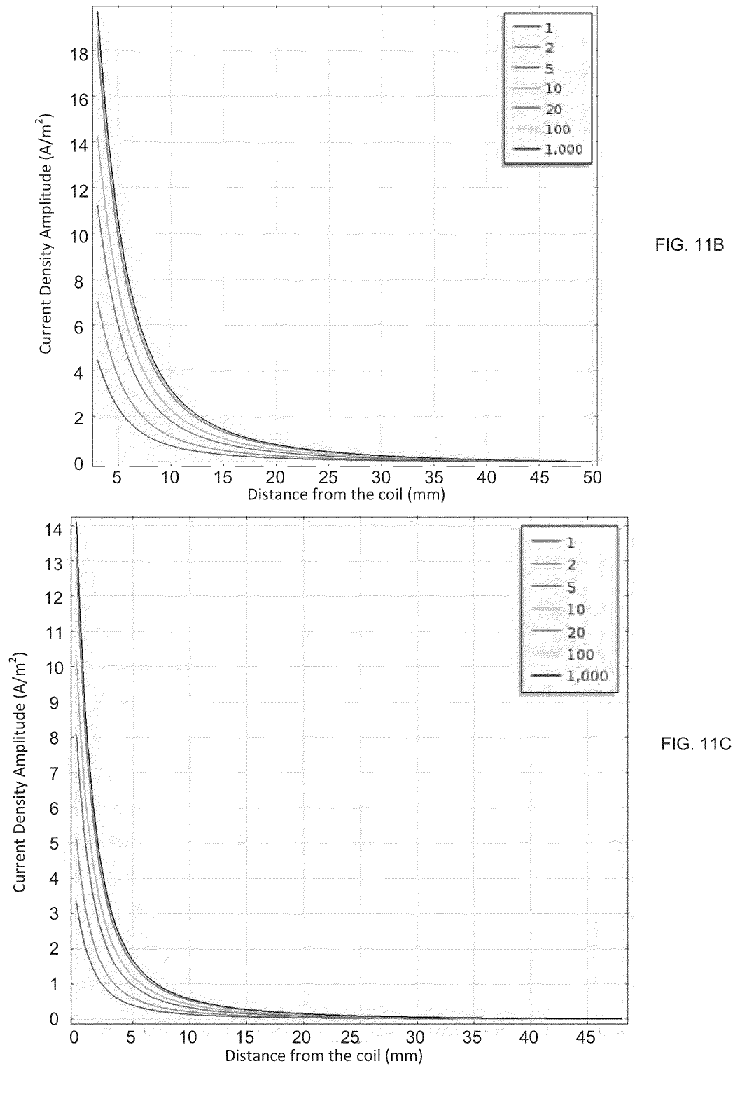

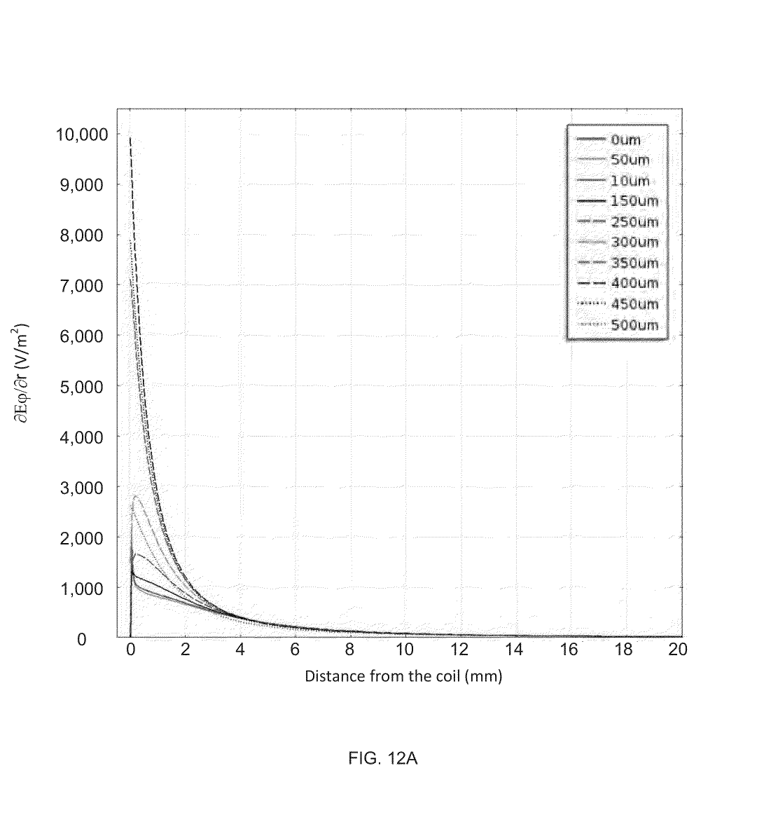

As shown in FIGS. 11A, 11B, 11C, the current densities norm increase non-linearly with the magnetic permeability for all coils 400, 600, and 800, respectively.

Effects of Coil Diameter on Spatial Gradient of the Electric Field.

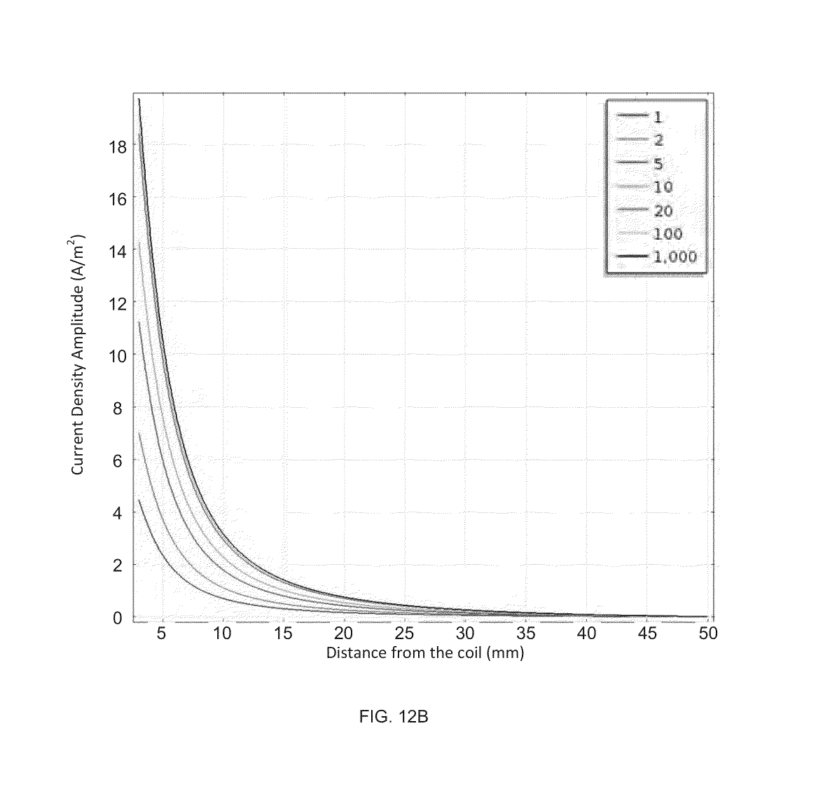

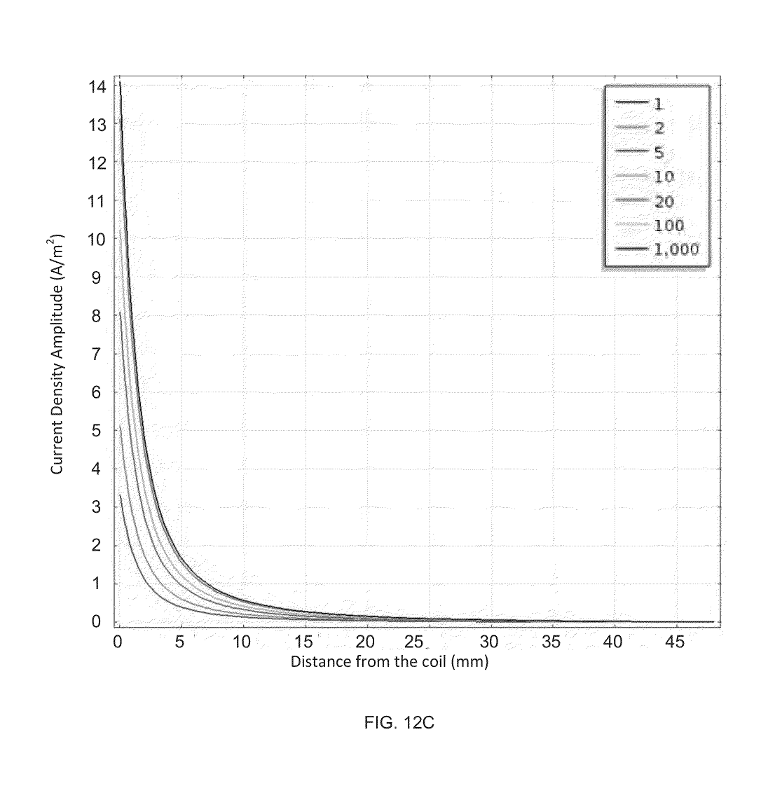

The spatial gradient of the coil's electric field, shown for coils 400, 600, and 800, respectively in FIGS. 12A, 12B, 12C, increases linearly with the size of the coil.

Inductance Calculation.

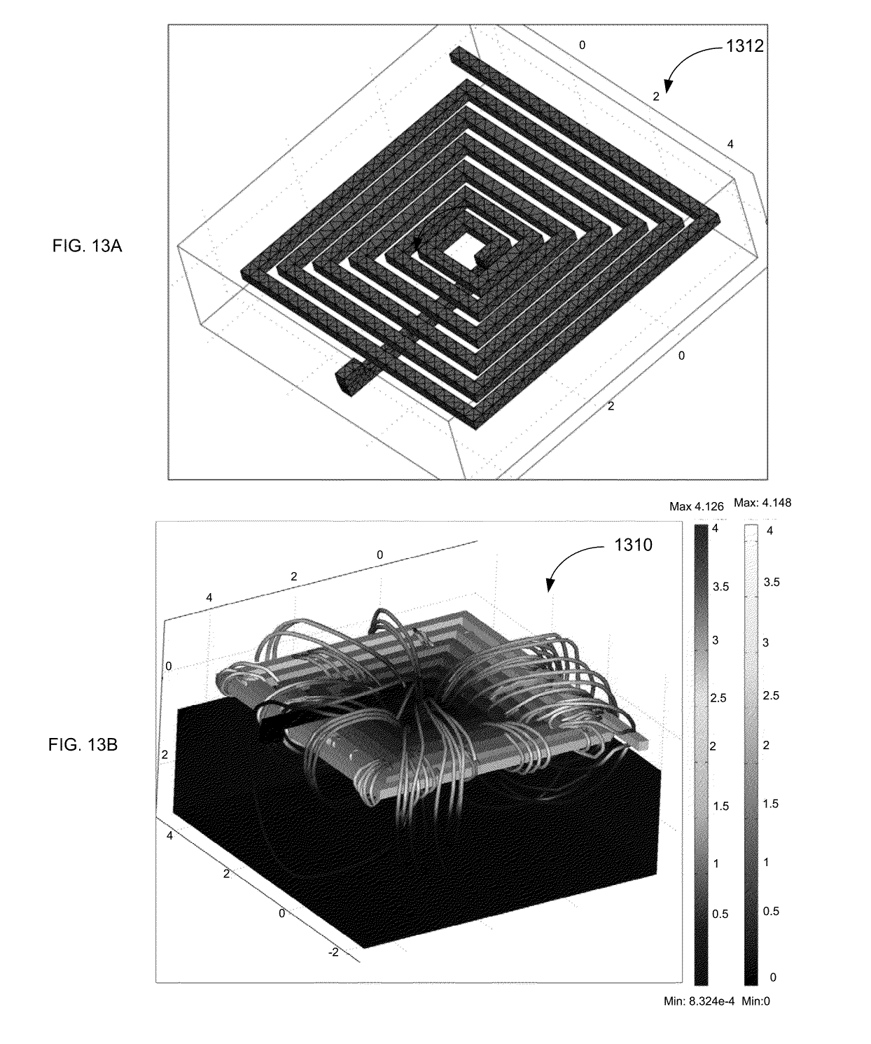

The calculation of inductance for different types of inductors can be found in related art. For planar spiral square coils, for example, it was shown that the optimal Q can be achieved when the OD/ID (the ratio of an outer diameter to an inner diameter) is about 5. Here, we considered such planar square coil 1310 (a finite-element model 1312 for which is shown in FIG. 13A, with FIG. 13B illustrating lines of magnetic flux and electric potential for such coil).

The total inductance has been estimated as the sum of the self and the positive and negative mutual inductances:

Lₛ=L₀+M₊+M₋ (26)

where Lₛ is the total inductance. L₀, is the sum of the self-inductances of all the straight segments, M₊ is the sum of all the positive mutual inductances or when the current flow in two parallel conductors is in the same direction and M₋ for currents in the opposite direction. The current on opposite sides of conductors are parallel to one another, whereas the currents in the adjacent segments, or components of a loop/turn of the coil, are orthogonal.

Given that neurons are typically sensitive to low frequencies (i.e., ˜10² Hz) where dispersion and loss mechanisms are not predominant as in the MHz or GHz frequency bands, one can take advantage of the symmetry of the coil turns in a solenoid and the fact that loop segments with orthogonal current have zero mutual inductance, or when the change in current in one segment does not induce a current in another nearby segment. The inductance can be well approximated by the sum of the self-inductance of adjacent segments and the mutual inductance between two nearby conductors in which current flows in opposite direction:

$${L}_{s} = {μ}_{0} {N}^{2} {C}_{1} \frac{l + i}{4} [ log ( {C}_{2} \frac{l + i}{l - i} ) + {C}_{3} \frac{l + i}{l - i} + {{C}_{4} ( \frac{l + i}{l - i} )}^{2} ] \qquad ( 27 )$$

This simple approximation has an error lower than 8% for s≦3w (i.e., spacing between segments is less or equal to three times the thickness of the traces), since smaller spacing between traces improves the magnetic coupling between loops and reduces the total length of the spiral.

The following nomenclature is used in Eq. (27):
N: total number of segments in the spiral
l: total length of the spiral (m)
i: inside diameter of the spiral (m)
w: width of the traces (m)
t: thickness of the traces (m)
t_{sub}: thickness of the substrate (m)
s: spacing between segments (m)
sᵤ: spacing between traces and underpass (m)
T: number of turns
G_{sub}: conductance per unit area of the substrate (S/m²)
X_{sub}: capacitance per unit area of the substrate (S/m²)
∈_{sub}: dielectric constant of the substrate
ρ is the resistivity of the traces (Ω-m)
μ the permeability of the traces (H/m)
μ₀ the permeability of vacuum or 4π 10⁻⁷ (H/m)
f the frequency (Hz)
k=0.50049
C₁=1.27
C₂=2.07
C₃=0.18
C₄=0.13

TABLE 2
Parameters used in estimation of inductance.

| Parameters | Analytical Equation |
|---|---|
| Lₛ | eq. (4) |
| Rₛ | $\frac{ρ l}{w t}$ |
| Cₛ | $\frac{T {w}^{2} {ε}_{ox}}{{s}_{u}}$ |
| Cₒₓ | $\frac{w l {ε}_{sub} }{2 {t}_{sub}}$ |
| C_{sub} | $\frac{w l {X}_{sub} }{2}$ |
| R_{sub} | $\frac{2 }{l w {G}_{sub}}$ |

Discussion.

Although neuronal stimulation using electrical techniques has proven quite useful for some applications, the disclosure of this invention is focused on μMS because it offers several advantages over purely electrical methods. In order for an electrode to generate current, the electrode needs to be in contact with a conductive media (such as, for example, excitable tissue). A magnetic coil can induce a current from a distance (for example, through an insulating layer). Unlike TMS, the μMS coils of the present invention can be placed within or immediately adjacent to the neural tissue, thereby greatly reducing the power needed to evoke neuronal activity. This disclosure explores the effects of coil geometry, stimulation frequency, and magnetic permeability on the resulting current density norm. The presented results demonstrate that: (a) μMS does not require charge-balanced stimulation waveforms as in electrical stimulation, (b) microcoils of different sizes and core material permeabilities produce non-linear changes in the electric field induced in the tissue, and (c) the ideal shape for a stimulation pulse is one with maximum slope or slew rate, at least from an energy efficiency point of view, though in vivo experiments must yet confirm that such a pulse will indeed excite neural tissue.

The distance at which certain current-density norms peak has been observed to scale in approximate proportion to the diameter of the coil. For instance, the peak for a distance of 300 μm from the 400 μm OD coil (Which is the distance this μMS coil activates retinal ganglion cells, albeit at a slightly lower, 70 kHz, frequency) is 18 A/m². For a 200 μm OD coil, the same peak current density norm appears at approximately 150 μm, and for the 100 μm coil, the peak occurs at 75 μm OD.

In electrical stimulation, using a bipolar electrode pair, the ‘cathode’ or source, injects a current that depolarizes the neuronal membrane, driving the potential towards a more positive E that can generate an action potential. At the ‘anode’ or sink, the axonal membrane potential is hyperpolarized towards a more negative potential that can inhibit the propagation of an action potential. The principal issue in the design of electrical stimulation pulses is that charges may accumulate over time, producing electrolysis byproducts that can damage or produce inflammation of neural tissue. In contrast, neither sinks nor sources exist when a current is induced by a magnetic field. This current consists of a rotating field that mirrors the current flow in the coil. The property of having no net sources or sinks as derived from the Maxwell Equations insures that, with μMS, there is never a net charge buildup.

Microelectrodes deliver currents into the surrounding tissue by means of resistive coupling at the point of tissue-electrode contact. The approach illustrated in this disclosure is to generate currents by means of inductive, rather than resistive, coupling. As demonstrated by traditional macroscopic TMS, magnetically-induced currents can induce neural activity. However, employing TMS in standard medical therapy is hindered by several technical and practical limitations, such as large device sizes, low spatial control and potential patient discomfort. TMS requires the coils to be relatively distant from the neural tissue, as opposed to μMS, where coils can be placed in close proximity to the target tissue. The large distances involved in using TMS therapeutically has three major implications. First, TMS requires an extremely high current to drive the coils sufficiently to elicit neural activity. The current-driving sources utilized in TMS are many orders of magnitude higher than those of μMS, and thus have significantly higher power requirements. Second, TMS generates strong magnetic fields that stimulate broad cortical areas; offering limited spatial control over the elicited activity. In contrast, μMS coils can be placed in close proximity to the neural elements, which allows more precise spatial control. While TMS is non-invasive, TMS can be uncomfortable for the patient because the induced current often activates the muscle overlying the skull, which is not with a factor in the use of μMS (although μMS is an invasive technology).

Theoretical studies of TMS include characterizing the electrical fields produced in neural tissue by magnetic stimulation with the use of coils in the centimeter-diameter range (i.e., 2, 3.5 and 5 cm). The results of such TMS simulation has shown only a limited ability to confine the induced current to a small brain area, and that such limitations could be at least partially overcome by more effective coil positioning and/or assembly. Other theoretical TMS studies make use of Eaton's model, comparing the effects of a 6-cm coil vs. two 5-cm coils arranged in a figure-eight geometry (Eaton, H., 1992, “Electric field induced in a spherical volume conductor from arbitrary coils: application to magnetic stimulation and MEG,” Medical & biological engineering & computing, 30(4), pp. 433-440). The latter was shown to generate a more symmetrical, focal, and deeper E-field distribution than did the single coil. Eaton's model showed that a current of 3,700 A with a 200-μs rise time, running through a single winding, would induce a 18.75 V/m peak E-field, which is the theoretical threshold for exciting a 10-μm nerve fiber. To achieve the same degree of stimulation, the figure-eight coil needs a 30% lower peak current, and the area of cortex thus stimulated is more focused. Thus, the results of Eaton's model (albeit scaled down in the case of the μMS coils) raises the expectation that the figure-eight geometry will allow us to reach deeper into the tissue, while achieving more focal stimulation than with TMS.

The use of a magnetic field to generate current flow in tissue is extremely inefficient from an energy standpoint with respect to its capacity for inducing electrical fields or currents in a medium. The idea of the present invention is that the opposite may prove true for neural stimulation at the microscopic level. One important difference between electric and magnetic field is that the magnitude of the latter is well known to fall off much more rapidly with distance (e.g., cubic vs. quadratic laws for electric vs. magnetic dipoles in empty space). The present idea stems from a consideration that the field gradient, rather than the field strength, is primarily responsible for neural stimulation. The FEM simulations confirm that a very high (i.e., 83 V/m²) electric field gradient in the physiological solution at the distances of interest, between 50 μm and 125 μm from the μMS coil. On the other hand, the gradient is much smaller than the one used, for example, in our simulations involving relatively large (i.e., 6 mm) electrodes (see, for example, Shawki, M. M., Elbelbesy, M. A., Shalaby, T. E., kotb, M. A., and Youssef, Y. S., 2010, “Studies On The Electric Field Distribution Using Different Electrode Shapes For Electrochemotherapy,” World Congress on Medical Physics and Biomedical Engineering, Sep. 7-12, 2009, Munich, Germany, O. Dassel, and W. C. Schlegel, eds., Springer Berlin Heidelberg, pp. 252-255).

Recently we demonstrated that μMS is capable of eliciting neuronal activation, both in vitro and in vivo in inferior colliculus neurons. The in vitro experiments, performed in a retinal cell preparation, demonstrated that action potentials could be elicited by μMS. It was also shown that neuronal activation was amplitude-dependent, whereby higher simulation amplitudes resulted in more intense activation, and that varying the orientation of the coils relative to the neural substrate resulted in different activation patterns. Perpendicular orientations of the coil resulted in minimal activation, whereas parallel orientations resulted in maximal activation. In the in vivo experiments, it was demonstrated that μMS of the dorsal cochlear nucleus resulted in neuronal activity in the inferior colliculus. Hence, μMS can elicit neuronal activation within an interconnected neural circuit and is not restricted to modulation of local circuitry only. Yet despite these results, a number of key issues need yet to be addressed before μMS can become useful in fundamental research or in translational chronic neuromodulation therapies.

One primary issue relates to the amount of energy needed to sustain neuromodulatory effects using μMS. The energy required for μMS simulation can be considerably reduced by employing at least these three strategies: (1) optimizing the stimulatory pulse shape, (2) designing coils with high permeability, and (3) recovering unused energy. First, energy could be optimized by using a more efficient pulse sequence. Traditional electrical stimulation pulse shapes are inadequate for μMS stimulation since most of the energy is lost due to Faraday induction (Eq. 8). Therefore, shorter pulses are preferable since less energy will be dissipated and larger current densities can be induced in the tissue. However, there are limitations in the duration of the excitation pulse since simulations suggest that with sinusoidal stimuli, the activation threshold increases monotonically with frequency when the stimulation plateau of a few kHz is exceeded. In so optimizing stimulation sequences, one should endeavor to minimize the power and maximize the slope while still retaining the ability to activate neural tissue. Second, micro coils could be specifically designed for this application. For example, the long solenoid design used here optimizes the Q-factor but renders the bulk of the magnetic energy inaccessible. An alternative is the use of multiple, slimmer coils (i.e., spiral, as discussed above) or by using the principle of field summation. The dimensions of the coil can further be reduced using advanced MEMS construction techniques and by generating fields with even higher gradients. However, there are limitations on the magnitude of the magnetic field flux density obtainable by increasing size because of the aforementioned limits on current densities for various metals. Finally, inductors store energy (Eq. 1) in the magnetic flux density, and it is well-known that this energy can be recovered. For example, the energy can be extracted from the magnetic flux density of the inductor by disconnecting the generator and connecting the coils to a capacitor. Therefore, it may be possible to recover unused energy in an implanted μMS system and thus extend battery life. The ability to recover energy with a μMS-based inductor is not possible with conventional stimulation employing a microelectrode, such as with DBS, where much of the power is dissipated as thermal energy.

The peak instantaneous power (PIP) in DBS is typically about 3 mW, whereas in previous microscopic magnetic stimulations of ganglion cells in the retina the PIP was approximately 5 W. A decrease in PIP of about three orders of magnitude may therefore be needed for effective future potential FEF replacement. Approximately one order of magnitude of improvement can be achieved by changing the material of the microcoil's core from air to high magnetic permeability materials. Another order of magnitude can be achieved by decreasing the distance separating the coil and neurons, for example decreasing such distance from 300 μm to less than 10 μm (according to the results illustrated in FIG. 4B). Hence, in order to get closer to neurons the insulator of the coil has to be made thinner, which can be achieved with thin film dielectric coatings. A further order of magnitude of reduction or more can be achieved by increasing the slew rate or the frequency of the stimulation waveform driving the coil. However, this increase maybe curtailed by the effective limitation on the current densities allowed by the conductor used in the coils, the corresponding increases in stress and vibration of the coils that could be alleviated by an advanced coil design (such as that of FIG. 2D) and the lower sensitivity of neurons to respond to high frequency time-varying magnetic fields. Driving currents with a magnetic field rather than an electric field (such as in the case of FES) may increase the sensitivity of excitable tissue. A decrease in coil size may allow for increase neuronal sensitivity as the gradient of current density increases. Finally, PIP maybe reduced by utilizing “ideal” pulse stimuli, which maybe substantially different than FES for neuronal activation, because for in magnetic stimulation only the rising or falling edges induce currents in the tissue whereas constant currents do not induce any current. For instance, a positive pulse followed by a negative pulse induced in the tissue can be produced by a triangular current signal in the coil. Furthermore, power can be recycled by recovering part of the energy stored in the magnetic flux density. Since the instantaneous power driven in the inductor is not dissipated as heat, but stored in a magnetic field in its core, and the energy can be recovered. When the current in the coil increases driven by the current generator then extra energy is stored in the magnetic field and vice versa when the current decreases then magnetic field energy can be recovered as current. This can be accomplished by a LC circuit or a circuit containing the coil and a capacitor. When the coil starts to replenish by releasing the energy it stored in its magnetic field it will send a current to charge the capacitor, which will be then ready for the next stimulation by releasing energy from the electric field of the charged capacitor starting a current which stores energy in the magnetic field of the coil.

As demonstrated by the traditional macroscopic TMS, the magnetically-induced currents can induce neural activity. However, TMS requires that the coils be typically between 15-30 mm away from the neural tissue, as opposed to μMS, where the coils can be placed in close proximity to the target neurons. TMS stimulates widespread cortical areas, offering limited spatial control over the elicited activity. These limitations make TMS inherently unsuitable for chronic prosthetic applications. Theoretical studies of TMS include the computation of the electric fields produced in neural tissue by magnetic stimulation using coils with different diameters (for example, 2, 3.5 and 5 cm). These results from TMS simulations showed a limited opportunity to concentrate the current spread into a small brain area, and that such concentration can possibly be at least partially achieved with more effective coil positioning and/or assembly.

Coil sets for microscopic magnetic stimulation are not simply TMS coils scaled down in size: the coils used in the present invention have different configurations (see, for example, FIGS. 2A, 2B, 2C, 2D, 2E, 2F) and are covered with the biocompatible coatings (and not simply an insulator material). Embodiments of the coil sets of the invention also differ as far as the shape of the individual coil is concerned (square, spiral, solenoid). Despite Nicola Tesla's own fascination with flat spiral coils, we have studied solenoid type since it is the most common type of microscopic surface mount components: solenoid coil allows for a closer reach of the magnetic field to the tissue, having no terminals on the way.

The reduction of coil size has two important advantages: power reduction and reduction in stimulation threshold due to the increase in spatial gradient of the magnetic field. Studies conducted with the use of isolated frog sciatic nerves have shown that, in an action potential, the contribution of the magnetic field from the current inside the membrane is approximately two orders of magnitude greater than the field induced by the currents outside the membrane. Similarly, neural stimulation depends on the spatial distribution of the magnetic field a well-known effect in MRI due to the spatial waveform distribution of the magnetic flux field used for imaging.

According to embodiments of the invention, the inductance value is varied and, in particularly, increased by using coil-core materials such as magnetic permalloys, or nickel (80%)-iron (20%) alloys to impart, on the coil, high permeability and fast response. Other materials for fabrication of microcoil core include alumina, alumina ceramic, amorphous, ceramic, dust, ferrite, iron, metal composite, metal dust, molybdenum permalloy (MPP), phenolic, polymer, powdered iron. However, as a result of the use of core materials different from air, hysteresis or eddy current losses may occur. Eddy current losses may also occur at microcoil terminals reducing the axial component of the magnetic field produced by the coil. Different core types need to be malleable to micromachining in order to be used in microcoils.

Capacitive coupling between terminals and tissue may occur depending on size, type of coating and amplitude of voltage applied to the microcoil. For the purposes of the present invention, however, capacitive coupling was not a mechanism behind microscopic magnetic stimulation since the retinal studies showed a decrease in stimulation sensitivity or an increase in the stimulation threshold when the terminals were directly facing the tissue.

Inductive coupling (and calculation of inductance) has been discussed above as an important phenomena that needs to be taken into account especially when working with multiple-coil configurations (such as those of FIGS. 2A through 2F). In multiple MEMS coils configurations, the increase of magnetic energy in the tissue can be improved by using appropriate 3D stacking procedures on the packaged coils by following the opposite of traditional coil coupling designs. For example, loops or turns are usually perfectly aligned to each other to trap the magnetic energy inside, therefore it is possible instead to shift the axis of adjacent loops to help the magnetic field escape sideways.

As embodiments of the present invention includes driving the μMS coils at high frequencies KHz to MHz range, an important characteristic is the self-resonance of the microcoil that limits the use of the coil as an inductor and it is strongly influenced by its geometrical design.

Generally, an embodiment of the invention may include a microcoil array with either multiple driving sources (corresponding to either individual coils or groups of coils from the microcoil array) or a single driving source for an array here that has the ability of steering the equivalent or equivalent magnetic dipoles in various directions to generate the spatial gradient of the electric field aligned with a nerve fiber, generating thereby an action potential associated with such nerve fiber.

In one embodiment of the invention, low cost compact class-D amplifiers (developed using CMOS technology) could be adapted for μMS stimulation to include a switched capacitor design that would allow for the recovery of the energy unused for neuronal stimulation but stored in the inductor. Bonmassar et al. have proven the μMS to be an effective in-vitro approach to activating local neural circuitry of the retina. Related work using electric stimulation of retinal ganglion cells demonstrated that the spiking response consisted of two components: an early phase (<1 ms) that results from direct activation of the ganglion cell and (2) a late phase (tens to hundreds of milliseconds) that results from the activation of neurons presynaptic to the ganglion cell. Micro-magnetic stimulation is also capable of eliciting both the early and late responses as observed with electrical stimulation. Interestingly, different spatial orientations of the coils relative to the neuronal tissue can be used to generate specific neural responses. These data indicate that μMS is operable to stimulate neuronal circuitry identical to electrical stimulation and that this response can be effectively “tuned” by the positioning of the microcoils relative to the tissue. This feature of μMS would be of interest to the researchers interested in studying the effect of differing stimuli parameters on downstream responses.

It is appreciated that the process of driving microcoil(s) of a neural stimulator of the invention may require the employment of a processor controlled by instructions stored in a memory to effectuate, for example, a control of parameters of a current pulse used to drive a microcoil, as a function of time. The memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

References made throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, it is to be understood that no single drawing is intended or even capable to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

The invention as recited in claims appended to this disclosure is intended to be assessed in light of the disclosure as a whole.

While the invention is described through the above-described exemplary embodiments, it will be understood by those of ordinary skill in the art that modifications to, and variations of, the illustrated embodiments may be made without departing from the inventive concepts disclosed herein. Disclosed aspects, or portions of these aspects, may be combined in ways not listed above. Accordingly, the invention should not be viewed as being limited to the disclosed embodiment(s).

What is claimed is:

1. A method for operating an array of inductive microelements to affect neural activity of biological tissue, the method comprising:
disposing the array of multiple magnetic coils at a first distance not exceeding 900 microns from the biological tissue,
magnetically inducing, in the tissue, an induced current having a peak value of norm of current density, by passing through said magnetic microcoil a driving current including a driving current pulse having an amplitude and a duration that are defined based on empirical data representing a level of neuronal firing threshold in said biological tissue,
wherein said induced current formed when said magnetic microcoil is driven with a driving current having an amplitude not exceeding 100 amperes;
and
wherein a magnetic coil has geometrical parameters chosen to define said peak value, at a point located at a second distance from said magnetic microcoil, to be proportional to an outer diameter of said magnetic microcoil.

2. A method according to claim 1, wherein said magnetically inducing includes
passing through said magnetic microcoil a pulse of the driving current, said pulse of the driving current being asymmetric as a function of time to cause an induced current pulse that is defined by any of
(i) a front rising edge, a slope of which is characterized by a first rate, a rear falling edge, a slope of which is characterized by a second rate, the first rate being higher than the second rate; and
(ii) a front rising edge, a slope of which is characterized by a first rate, a rear falling edge, a slope of which is characterized by a second rate, the first rate being lower than the second rate;
and
a corresponding induced current pulse duration,
wherein one of said front rising and rear falling edges exceeds said level of neuronal firing threshold and another of said front rising and rear falling edges is below said level of neuronal firing threshold.

3. A method according to claim 1, wherein said magnetically inducing includes passing through said magnetic microcoil a pulse of the driving current represented by a function of time defined to minimize power consumption in said magnetic microcoil.

4. A method according to claim 1, wherein said magnetically inducing includes passing through said microcoil, operably connected to electronic circuitry, a pulse of the driving current that causes a formation, in said tissue, of electrical field a modulus of which is represented, as a function of time, by two rectangular pulses of opposite signs.

5. A method according to claim 4, further comprising storing electromagnetic energy, that has been drained from said microcoil during a second rectangular pulse of said electrical field, in a capacitor of said electronic circuitry.

6. A method according to claim 1,
wherein said disposing includes disposing multiple pairs of microcoils in a spatial configuration that
defines that a first axis of a first microcoil and a second axis of a second microcoils in a pair of microcoils are parallel to one another, and
ensures that a magnetic field, formed by the multiple pairs of microcoils when first microcoils are driven by respectively corresponding voltage inputs, is steerable in any direction in response to changing voltage values of said voltage inputs.

7. A method according to claim 6, wherein the disposing includes disposing pairs of microcoils such that first and second microcoils in a pair are coaxial with one another.

8. A method according to claim 6, wherein the disposing includes disposing the first and second microcoils of a pair such that directions of winding respectively corresponding to said first and second microcoils are opposite to one another.

9. A method according to claim 6, wherein the disposing includes disposing a microcoil having a core material with magnetic permeability that is different from that of air.

10. A method for operating an array of inductive microelements to affect neural activity of biological tissue, the method comprising:
magnetically inducing, in said tissue, an induced current by passing through a magnetic microcoil from said array, disposed at a first distance not exceeding 900 microns from the tissue and operably connected with electronic circuitry, a pulse of driving current having an amplitude and a duration that are defined based on empirical data representing a level of neuronal firing threshold in said tissue,
and
storing electromagnetic energy, that has been drained from said microcoil during a time period corresponding to a falling slope of said pulse of driving current, in a capacitor of said electronic circuitry.

11. A method according to claim 10, wherein said magnetically inducing includes passing through said magnetic microcoil a pulse of driving current the amplitude of which does not exceed 100 amperes.

12. A method according to claim 10, wherein said magnetically inducing includes passing said pulse of driving current represented by a function of time defined to minimize power consumption in said magnetic microcoil.

13. A method according to claim 10, wherein said magnetically inducing includes passing a triangular pulse of driving current having a front edge rising at a first rate and a rear edge falling at a second rate, the first and second rates being different.

14. A method according to claim 10, wherein said magnetically inducing said induced current includes magnetically inducing an induced current a peak value of norm of density of which is proportional to an outer diameter of said magnetic microcoil.

15. A method according to claim 10, wherein said magnetically inducing includes passing driving current through multiple pairs of magnetic microcoils, of said array, that are spatially configured such that
a first axis of a first magnetic microcoil and a second axis of a second magnetic microcoil in a pair are parallel to one another, and
a magnetic field, formed by the multiple pairs of microcoils when first microcoils are driven by respectively corresponding voltage inputs, is steerable in any direction in response to changes in voltage inputs applied, respectively, to said first microcoils,
and
generating action potential in said tissue caused by said magnetic field.

16. A method for operating an array of inductive microelements to affect neural activity of biological tissue, the method comprising:
magnetically inducing, in said tissue, an induced current by passing through multiple pairs of magnetic microcoils, disposed at a first distance from the tissue, at least one pulse of driving current having an amplitude and a duration that are defined based on empirical data representing a level of neuronal firing threshold in said tissue,
said magnetic microcoils being spatially configured such that a first axis of a first magnetic microcoil and a second axis of a second magnetic microcoil in a pair are parallel to one another,
said at least one pulse of driving current having a front edge rising at a first rate and a rear edge falling at a second rate, the first and second rates being different;
and
storing electromagnetic energy, that has been drained from said microcoil during a time period corresponding to a falling slope of said at least one pulse of driving current, in a capacitor of said electronic circuitry.
a magnetic field, formed by the multiple pairs of microcoils when first microcoils are driven by respectively corresponding voltage inputs, is steerable in any direction in response to changes in voltage inputs applied, respectively, to said first microcoils.

17. A method according to claim 16, wherein said magnetically inducing includes passing said pulse of driving current through said multiple pairs of magnetic microcoils that are spatially oriented to cause a magnetic field, formed by the multiple pairs of microcoils when first microcoils are driven by respectively corresponding voltage inputs, is steerable in any direction in response to a change of a voltage input applied to said first microcoils

18. A method according to claim 16, wherein said magnetically inducing said induced current includes magnetically inducing an induced current a peak value of norm of density of which is proportional to an outer diameter of said magnetic microcoil.
